SEQUENCE LISTING (4 sequences)

SEQ ID NO: 1 - protein, 26 aa, Staphylococcus aureus
MAQDIISTIGDLVKWIIDTVNFKTKK

SEQ ID NO: 2 - protein, 70 aa, Trichoderma reesei [sequence omitted]

SEQ ID NO: 3 - protein, 71 aa, Trichoderma reesei
AVCPTGLFSNPLCCATNVLDLIGVDCKTPTIAVDTGAIFQAHCASKGSKPLCCVAPVADQALLCQKAIGTF

SEQ ID NO: 4 - protein, 75 aa, Trichoderma reesei
SNGNGNVCPPGLFSNPQCCATQVLGLIGLDCKVPSQNVYDGTDFRNVCAKTGAQPLCCVAPVAGQALLCQTAVGA

(19) United States
(12) Patent Application Publication
(10) Pub. No.: US 2004/0234952 A1
(43) Pub. Date: Nov. 25, 2004

(54) Process for identifying microoraganisms by means of mass spectrometry

(76) Inventors: Wibke Kallow, Berlin (DE); Marcel Erhard, Potsdam (DE); Ralf Dieckmann, Berlin (DE); Stefan Sauermann, Potsdam (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/752,224

(22) Filed: Jan. 7, 2004

Related U.S. Application Data
(60) Provisional application No. 60/438,315, filed on Jan. 7, 2003.

(30) Foreign Application Priority Data
Jan. 7, 2003 (DE) 103 00 743.1

(51) Int. Cl. C12Q 1/70; G01N 33/53; G01N 33/569; G01N 33/554

(52) U.S. Cl. 435/5; 435/7.31; 435/7.32; 702/19

(57) ABSTRACT

The invention relates to a process for identifying microorganisms by means of mass spectrometry, especially by means of MALDI-TOF-US. According to the invention, a data base (DB) is used that comprises synthetic reference spectra (REFₛ) of known microorganisms, which are formed by combination of a number, reduced relative to natural mass spectra (REF_{N}), of signals (S) that are specific to the respective microorganism, as well as difference spectra (DIF), which are formed by offsetting in each case two synthetic reference spectra (REF_{S}) of the known microorganisms. The process also comprises a first analysis step, in which a similarity analysis of a sample mass spectrum (SAM) of a microorganism that is to be identified is performed with synthetic reference spectra (REF_{S}) that are contained in data base (DB), and a second analysis step, in which a similarity analysis of sample spectrum (SAM) is performed with at least a portion of difference spectra (DIF) that are contained in data base (DB).

This application claims the benefit of the filing date of U.S. Provisional Application Ser. No. 60/438,315 filed Jan. 7, 2003.

The invention relates to a process for identifying microorganisms by means of mass spectrometry, especially by means of Matrix-Assisted-Laser-Desorption-Ionization Time-of-Flight Mass Spectrometry (MALDI-TOF-MS) with the features that are mentioned in claim 1 as well as a data base that is suitable for implementing the process and that can be used for the process according to claims 21 and 25.

A quick and reliable identification of microorganisms is of decisive importance in various areas of health services, for example the diagnosis of infections, as well as the food industry. The traditional identification by means of direct identification of bacteria first requires the cultivation of the microorganisms from a material sample that are to be identified. Then, microscopic and visual examination processes primarily provide preliminary assessments of the bacteria content as well as the micromorphology or the color properties of the clinical study sample. The identification of isolates up to the species level fairly often requires a subcultivation for the purpose of obtaining a pure culture. According to a classical identification process in medical microbiology, the so-called “colored series,” specific metabolic performance levels of the microorganism to be identified are detected with use of a suitable combination of differential media. The main drawback of the microbial process is its very high time requirement, especially for the cultivation.

More modem molecular-biological approaches, such as the PCR method (polymerase chain reaction) and the 16S-rRNA method, involve the genetic analysis of the previously isolated genome or certain ribonucleic acids. These processes have become greatly important because of their high sensitivity. They are burdened, however, by a considerable expense in personnel and equipment just like the microbiological characterization.

Also, infrared-spectroscopic processes are known in which oscillation spectra of intact cells (“fingerprint spectra”) are recorded in FTIR spectrometers and are adjusted with a data base with oscillation spectra of known microorganisms. This still young technology is still being developed and can now be implemented only by special proficient and experienced personnel, so that in practice, this approach has not yet been widely accepted.

With the so-called MALDI-TOF mass spectrometry, in recent years, a process has been developed that is also accessible to the analysis of biological macromolecules in contrast to the usual mass-spectrometric process. In the case of the MALDI-TOF-MS technology, the sample that is to be examined with a mostly crystalline organic compound, the so-called matrix, is added to a sample plate, whereby the sample is incorporated into the matrix crystals and brought into interaction with a laser beam. In this case, individual molecules of the sample from the sample carrier are desorbed and ionized. Then, the thus produced ions are accelerated in an electric field, and their time-of-flight is recorded until a detector is reached. Since the acceleration depends on the mass of an ionized molecule, the times of flight reflect the molecular masses that are present in the sample. The MALDI-TOF-MS technology is now used primarily in the field of protein analysis (“proteomics”) and in RNA and DNA analysis.

It was proposed, for example, in WO 98/09314, that MALDI-TOF mass spectrometry be used for identifying microorganisms analogously to the above-described infrared-spectroscopic fingerprint method. For this purpose, the MALDI-TOF mass spectrum of a cell extract or intact cells of the unknown microorganism is compared to the spectra of known organisms. The comparison of the sample spectrum with the reference spectra of the data base is generally carried out with computer support by means of statistical-mathematical algorithms, which were developed in so-called pattern recognition processes. The mass spectra become more and more similar as the kinship between the microorganisms increases and as the probability of a repetition of certain signals in the mass spectra increases. This has the result that the process is already associated with elevated uncertainty at higher classification levels, for example with genera or families, if classification on a lower level (for example the strain level) fails, for example since no reference spectrum of the strain in the data base is present. The process therefore has to rely on very extensive data bases with a number of representative reference spectra of known strains. To this is added the difficulty of discriminating a spectral noise background from true signals.

Known from DE 100 38 694 A and EP 1 253 622 A is a process for identifying microorganisms by means of MALDI-TOF-MS, in which the sample spectrum of the unknown microorganism is not adjusted with original mass spectra (“natural mass spectra”) but rather with so-called synthetic reference spectra. In this case, these are mass spectra that are combined from a reduced number of signals that are characteristic of the respective microorganism. These characteristic signals preferably comprise those that could be assigned to certain molecular components of the cell and/or that were selected by visual or computer-aided analysis as specific. Although this process, because of the section of signals and limitation of the spectral comparison to the selected signals, exhibits a considerably improved reliability relative to the conventional adjustment with the original spectra, the differentiation of very similar organisms in individual cases has proven difficult. This applies in particular for very closely related organisms.

The object of this invention is therefore to further develop the mass-spectroscopic process for identifying microorganisms with respect to a still further increased reliability. A data base that is suitable for the process is also to be made available.

This object is achieved by a process with the features of claim 1 as well as by a data base and its use according to claims 21 and 25.

The process according to the invention for identifying microorganisms by means of mass spectrometry provides that

(a) a data base is used, comprising

synthetic reference spectra of known microorganisms, which are formed by a combination of a number of signals that are specific to the respective microorganism that is reduced relative to natural mass spectra, as well as

difference spectra, which are formed by offsetting in each case two synthetic reference spectra of the known microorganisms,

(b) in a first analysis step, a similarity analysis of a sample mass spectrum of a microorganism to be identified is performed with the synthetic reference spectra that are contained in the data base, and

(c) in a second analysis step, a similarity analysis of the sample spectrum is performed with at least one portion of the difference spectra that are contained in the data base.

The process according to the invention is accordingly distinguished from that of the known process essentially by a second analysis step, in which the sample spectrum is compared to difference spectra that are calculated from the synthetic reference spectra in further detail in the described way. This additional step produces a significant increase in the reliability of the process and also makes possible in particular the differentiation of strongly related organisms.

The difference spectra are preferably formed by subtraction of two synthetic reference spectra from one another, whereby signals that are present in both the reference spectra that are offset with one another, are completely eliminated regardless of their intensities. Provision can also be made for further selecting and/or weighting signals that remain in a difference spectrum of a known microorganism after subtraction. This can take place, for example, in that an adjustment of the remaining signals with the natural mass spectra (original spectra) of this microorganism and/or with the natural mass spectra of the microorganism that is offset with the latter is carried out. In particular, for this purpose, the frequency and/or the intensity of the signal in the “individual” natural mass spectra can be examined in this respect. Moreover, it is advantageous also to analyze the specificity of the signal, i.e., the frequency and/or intensity of the signal in the natural spectra (“foreign spectra”) of the microorganism, whose synthetic reference spectrum was offset with that of the microorganism in question.

To generate, for example, a difference spectrum for Escherichia coli, which facilitates the discrimination of the nearly indistinguishable Shigella, the synthetic reference spectrum of Shigella sonnei is subtracted from that of E. coli, whereby only signals that are present in the synthetic reference spectrum of E. coli but not in that of S. sonnei remain. Then, each remaining signal is studied with respect to its frequency and/or intensity with the original spectra of E. coli and with respect to its frequency and/or intensity in the original spectra of S. sonnei. A signal that is frequently in the individual original spectra but is unspecific, since it also occurs in the foreign spectra of Shigella, is either completely eliminated from the difference spectrum or is provided with a relatively low weight for the second analysis step. In this way, difference spectra that allow a very reliable differentiation of one microorganism from a certain other one are produced.

In addition, provision can especially preferably be made for determining a number of synthetic reference spectra that are similar to the sample spectrum as a result of the first analysis step and for performing the similarity analysis of the second step only with the difference spectra that were obtained from the offsetting of the synthetic reference spectra that are determined to be similar. A delimitation of the suitable microorganisms is thus carried out in the first step such that in the subsequent second step, only an adjustment with the difference spectra of the organisms that are determined to be similar is carried out. For this purpose, not only an enormous time advantage is gained, but also random hits are reduced, which result from proteins with randomly corresponding masses that are not identical. In this connection, it may also be useful to use a data base that from the start only contains difference spectra of microorganisms that are similar to one another.

It is provided that synthetic mass spectra are used as reference spectra that are produced by the combination of a number, reduced relative to “natural” (not reduced) mass spectra, of signals that are specific to the respective microorganism. By the reduction of reference spectra to a comparatively small number of characteristic signals, a considerable reduction in data and information is achieved. By the data reduction, not only is memory saved, but also the amount of time spent for data transmissions, such that the process is also suitable in principle for an application via crosslinked data-processing units (for example, the Internet). Moreover, the information reduction of the reference spectra of the data base makes possible a considerable acceleration in the similarity analysis of the mass spectrum of the microorganism that is to be identified with the reference spectra, since the analysis can now be limited to a comparison of the signals that are contained in the reference spectra.

Compared to conventional processes in which a sample spectrum of the organism that is to be identified is adjusted with “natural” reference spectra, reliability of the process according to the invention, i.e., the probability of correctly classifying the unknown organism, is considerably increased. Herein, the most important advantage of the process can be seen. The elevated reliability can be attributed to the high concentration of specific information in the synthetic reference spectra, which is not overlapped by low-significance signals or noise. Also, in a possible failure in a classification on the strain level of a sample, for example since no reference spectrum of this strain is available in the data base, the process provides reliable identifications in higher classification levels, for example in genus or species levels. The sensitivity of the procedure according to the invention is also not impaired by differences in various spectra, which are caused by, for example, different cultivation conditions, different cell stages or deviating signal-noise ratios.

The small sample amount of the microorganism that is to be identified that is necessary, which can be obtained in shorter cultivation times, represents another advantage of the process. Furthermore, mixed cultures can also be analyzed, such that the cultivation of pure cultures can be eliminated.

The signals that are contained in the synthetic reference spectra can be distinguished in two categories. An especially advantageous configuration of the invention provides that the signals of a reference spectrum comprise at least one identified signal that was clearly attributed to a characterized molecular cell component of the respective microorganism. Cell components that are suitable for identifying a microorganism are, for example, specific peptides, proteins, ribonucleic acids and/or lipids. It has proven especially advantageous for bacteria to undertake a signal classification of a ribosomal protein, especially a protein of the large ribosome subunit. For bacteria, these are proteins of the so-called 50S subunit, and in fungi, the 60S subunit. Proteins represent a main component of microbial cells. This applies in particular for ribosome proteins that are constantly present independently of a development stage of the cell, a nutrient supply or other cultivation conditions and thus represent reliable signals in the mass spectra. In addition, amino acid sequences of analogous proteins of different species or even different strains are at least slightly different from one another. Consequently, the analogous proteins exhibit different masses and are suitable for their differentiation. The inventors were thus able, for example, for the first time to relate four mass signals in the mass spectrum of Escherichia coli (m/z=4365, 5381, 7276 and 5096) to proteins L36, L34 and L29 of the large 50S-ribosome subunit or to protein S22 of the small 30S-subunit. These signals are constant components in mass spectra of E. coli, but not of many other microorganisms. They are therefore especially suitable for identifying Escherichia coli and for incorporation into a synthetic reference spectrum for E. coli. In the case of fungi, moreover, quite especially structural proteins, especially hydrophobins, have proven their value for identification.

In another development of this invention, it is provided that the signals of a synthetic reference spectrum comprise, as a second signal category, at least one empirical signal, which was determined to be specific for a microorganism by comparison of a number of mass spectra of known microorganisms. In this case, these are signals whose origin, i.e., whose causative molecular cell components, is not known specifically, but are considered characteristic of a microorganism because of specific criteria. These criteria preferably comprise a minimum frequency that can be specified in advance for the occurrence of a signal in a number of mass spectra of the same microorganism as well as an average minimum intensity of the signal that can be specified in advance. In this case, the minimum frequency should be at least 50%, especially at least 70%, preferably at least 90%. In addition, the specificity of the signal is examined, i.e., the frequency with which the signal occurs in the natural mass spectra of other microorganisms, whereby the occurrence in the foreign spectra should be as rare as possible. The determination of the empirical signals by comparison of measured mass spectra can be performed visually, but preferably computer-supported. Corresponding algorithms (pattern recognition processes) are known and are not to be explained in more detail here. Moreover, it is conceivable also to subject the identified signals to follow-up monitoring using these criteria.

The number of signals of a reference spectrum can lie in a range of 1 to 50 and is advantageously 5 to 30. In many cases, in particular a number of 10 to 15 has proven adequate. It is also preferably provided that a signal of a synthetic reference spectrum is represented by only one coordinate pair. In this case, the coordinate pair consists of a mass or a mass-charge ratio as x-coordinates on the one hand and an absolute or relative intensity as y-coordinates on the other hand. Compared to “natural” mass spectra, which contain several 1000 data points, this means a considerable reduction in data.

For the adjustment of a sample spectrum with the synthetic reference spectra of the data base, weightings corresponding to their significance can advantageously be assigned to the individually identified and empirical signals of the reference spectra, whereby the above-mentioned criteria, i.e., frequency/intensity in the individual spectra and in that of the other reference organisms therein can be used. If this weighting is already performed in the stage of the synthetic reference spectra, a corresponding selection and/or weighting of the signals of the difference spectra can be eliminated under certain circumstances.

The invention also comprises a data base for implementing a process as well as its use for identifying microorganisms by means of mass spectrometry. The data base according to the invention comprises

(a) synthetic reference spectra of known microorganisms, containing a number of signals specific to the respective microorganism that is reduced relative to natural mass spectra, as well as

(b) difference spectra, resulting from an offsetting in each case of two synthetic reference spectra of the known microorganisms.

Moreover, it may also be advantageous to contain the natural reference spectra (original spectra) in the data base.

The process according to the invention can be implemented especially advantageously with the MALDI-TOF mass spectrometry. Accordingly, all mass spectra used are preferably MALDI-TOF mass spectra.

The invention is explained in more detail below in the embodiments based on the respective drawings. Here:

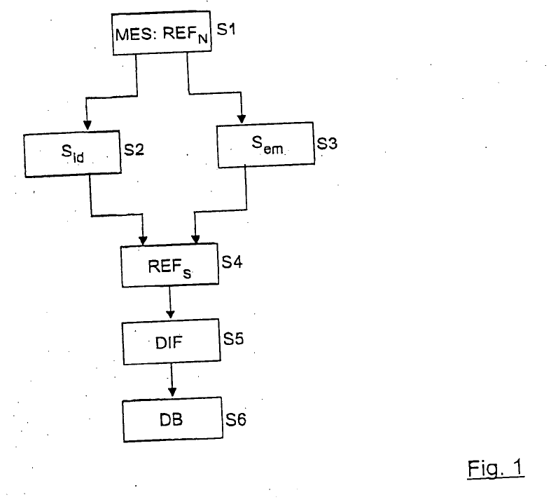

FIG. 1 shows a sequential diagram of the process for producing a data base according to the invention;

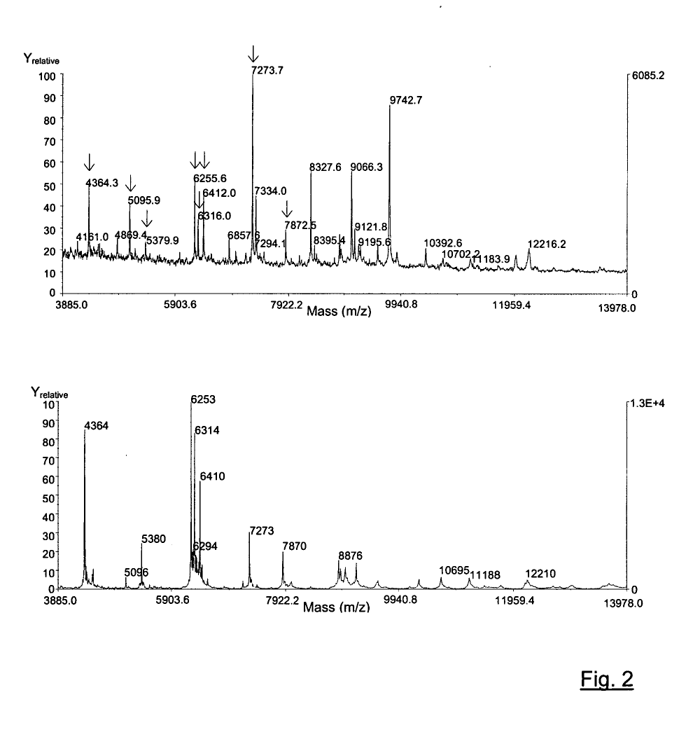

FIG. 2 shows MALDI-TOF mass spectra (original spectra) of Escherichia Coli and isolated ribosomes of Escherichia coli;

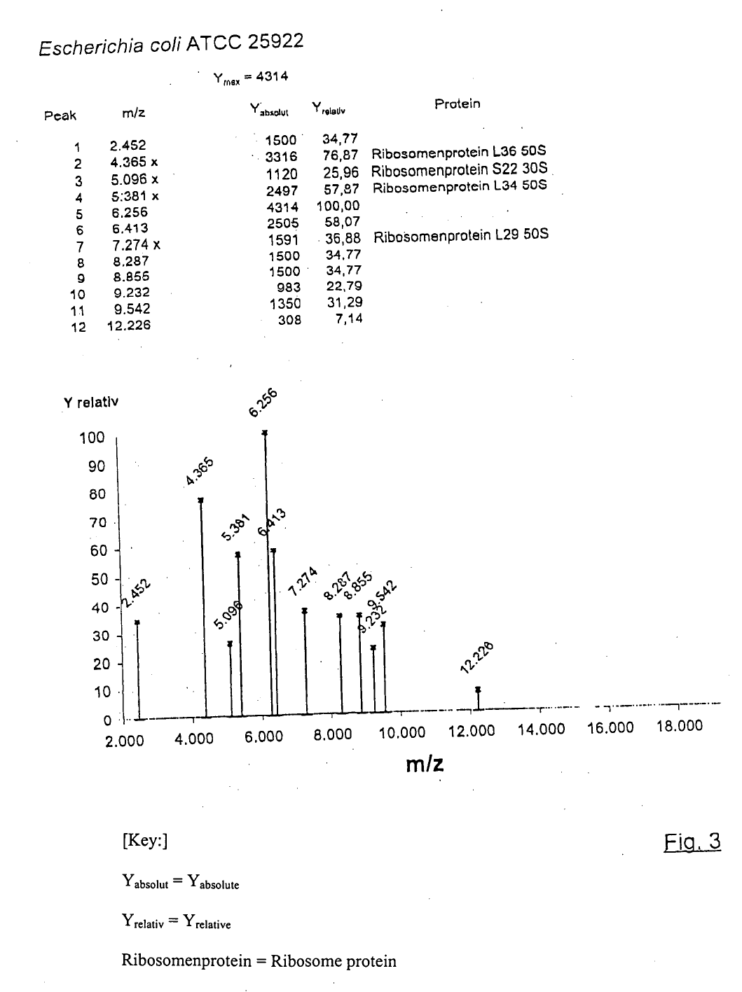

FIGS. 3 to 8 show synthetic reference spectra of various bacteria, especially for two Escherichia coli strains, two Klebsiella species, Pseudomonas aeruginosa and Staphylococcus aureus;

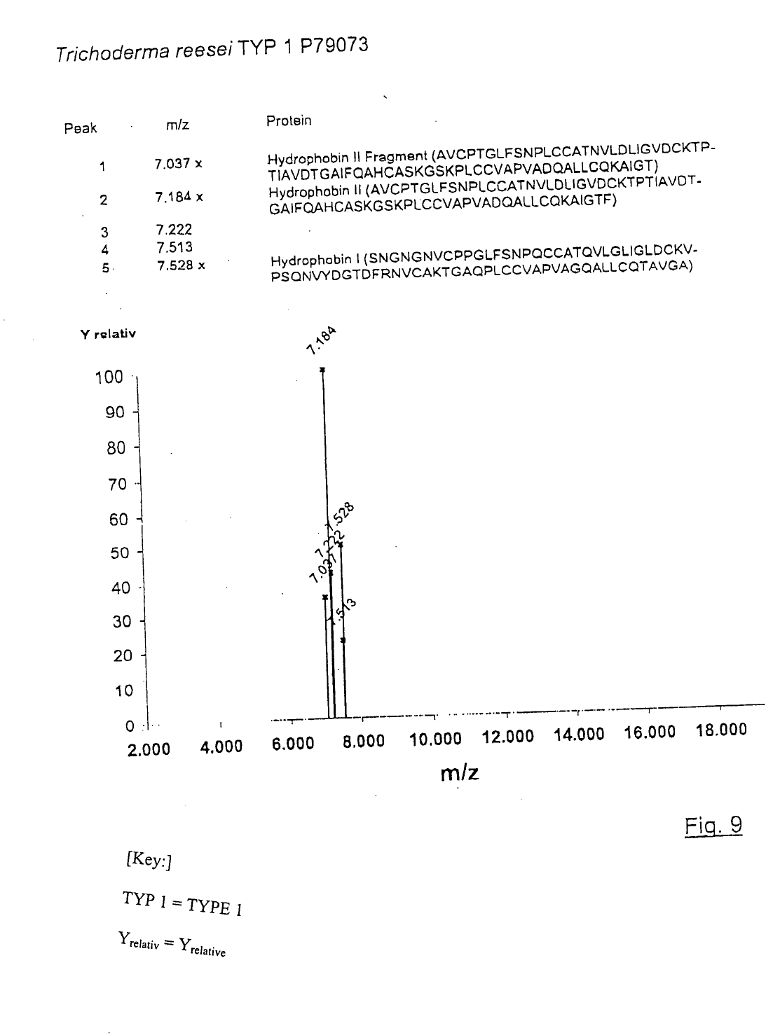

FIG. 9 shows a synthetic reference spectrum of the fungus Trichoderma reesei;

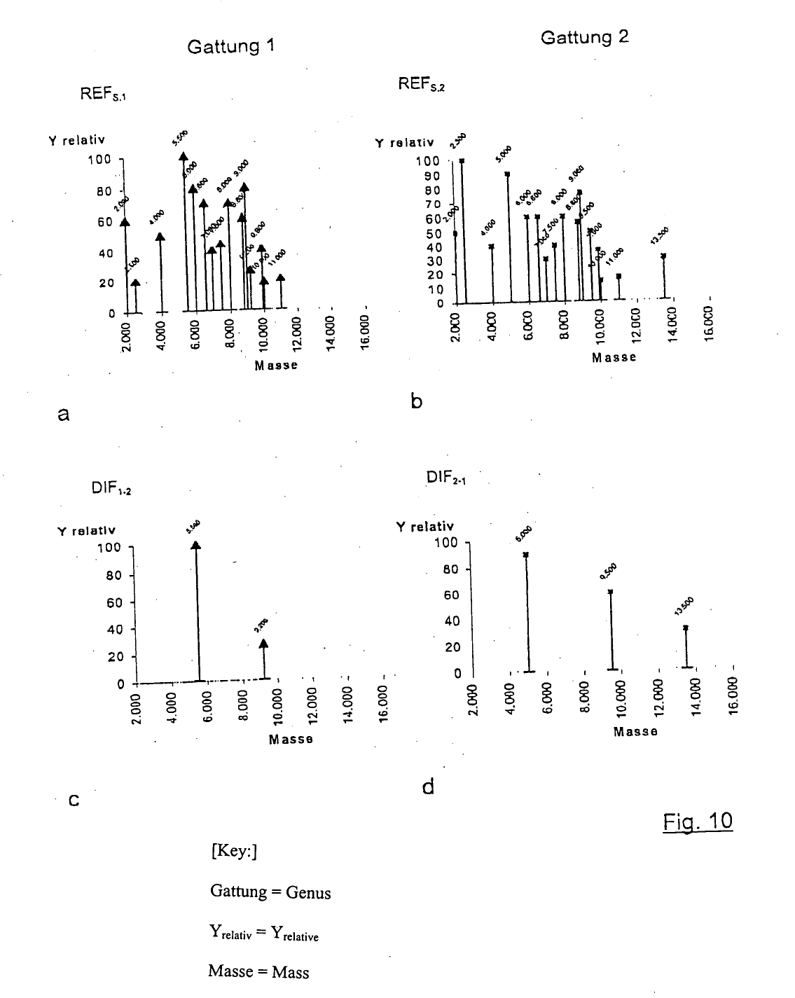

FIGS. 10a- show a production of two difference spectra; 10d

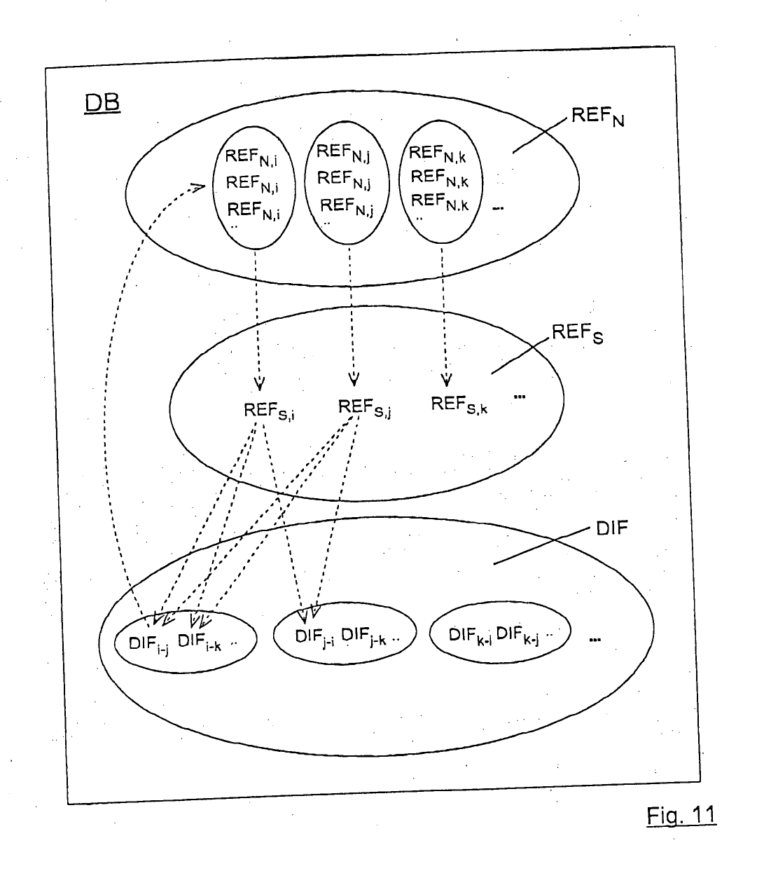

FIG. 11 shows a structure of the data base according to the invention; and

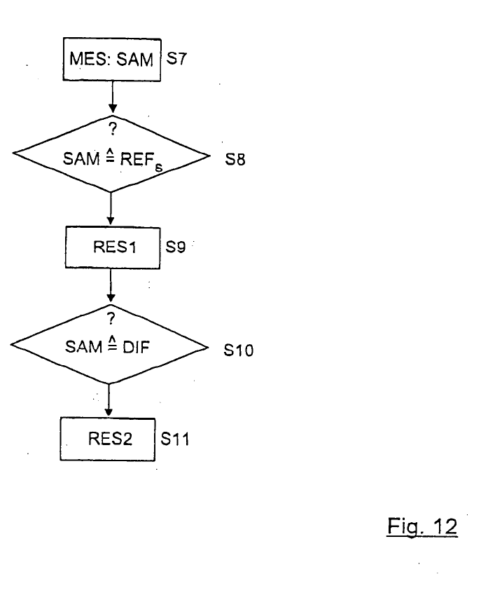

FIG. 12 shows a sequential diagram of the process for identifying a microorganism.

In a flow chart, FIG. 1 shows a typical course of the process according to the invention. In a first step S1, the largest possible number of natural reference spectra REF_{N}, i.e., original spectra of known microorganisms, is measured. In this connection, several details are provided below for sample preparation and for data acquisition. Sample Preparation and MALDI-TOF-Data Acquisition For a MALDI-TOF analysis, about 5 to 100 μg of wet cells or else 5 to 50 μg of dried cells of a known bacterium or a bacterium or fungus that is to be identified is required. A pretreatment of cells, for example cell decomposition, is not necessary. The wet cells can be transferred directly from an AGAR culture with a sterile inoculating loop to a sample plate that is also named a template. As an alternative, cells of a liquid culture that are centrifuged off can also be used. Then, the cells on the sample plate are mixed with 0.2 to 1 μl of a matrix solution. In a variant of this procedure, the wet cells can also be mixed with the matrix solution before their transfer to the sample plate and are transferred as a suspension. For the following measurements, a matrix solution that consists of 100 mg/ml of 2,5-dihydroxybenzoic acid in a mixture that consists of 50% acetonitrile and 50% water with 3% trifluoroacetic acid was used. Other known matrix solutions are also suitable. After the sample, which coincides with a crystal formation, is dried, the sample can be subjected directly to a MALDI-TOF mass-spectrometric analysis. For these measurements, each sample point of a sample plate in a conventional mass spectrometer was stimulated with about 50 to 300 laser pulses of a nitrogen laser with a wavelength of 337 nm. The acquisition of positive ionic mass spectra was carried out in the linear measurement mode in a mass range of 2000 to 20,000 m/z. A typical example of a positive MALDI-TOF-mass spectrum REF of Escherichia coli that is obtained in this way is shown in the upper portion of FIG. 2 in the mass range of about m/z=4,000 to 14,000.

In subsequent process step S2 (FIG. 1), an allocation of signals (peaks) to certain molecular cell components is performed. A possible procedure that uses known methods of biochemistry and molecular biology is briefly explained below in the example of ribosome proteins of large ribosome subunits.

Identification of Unknown Signals

A protein extract of a cell culture is separated by means of a 2D-gel electrophoresis. Suitable so-called protein spots are then subjected to a typical digestion in which the protein is enzymatically cleaved into small protein fragments. If antibodies against ribosomes are present, the relevant protein spots can also be recognized by an immunoassay (for example Western-Blot analysis). The protein fragments that are obtained by tryptic digestion are then separated by means of HPLC and sequenced or subjected directly to a so-called peptide-mass-fingerprint identification with subsequent PSD (post source decay). With the sequence fragments that are determined in this way, an attempt can then be made to identify the ribosome genes in a data base. If corresponding genes are present in the data base, the corresponding protein mass can be determined from the entire gene sequence. If a corresponding gene cannot be found in the data base, the entire gene, which codes for the corresponding ribosomal protein, must be isolated and sequenced with known agents of molecular biology, which are not to be explained in more detail here. If the gene sequence is known, the translation into the protein sequence and the determination of the theoretical protein mass follows. An examination of this theoretical mass can be carried out by the corresponding protein spot of the 2D-gel electrophoresis of a MALDI-TOF mass spectrometry being discarded. With deviations of the theoretical protein mass, modifications of the protein by MALDI-TOF analysis of the tryptic digestion can be noted.

For clarification, a mass spectrum of the 70S-ribosome of Escherichia coli, which was isolated by means of 2D-gel electrophoresis, is shown in FIG. 2 in the lower portion. The entire ribosome of a protein, which consists of the large 50S-subunit and the small 30S-subunit, is referred to as 70S. In turn, both subunits consist of a number of proteins that are referred to with the letter L (for large) and the letter S (for small). It is readily evident that a number of the most intensive signals from the spectrum of Escherichia coli ATCC 25922 (cf. FIG. 2 of the upper portion) can be attributed to ribosomal proteins. Corresponding signals are identified with arrows in the figure. For the first time, the inventors were able to relate three of these signals to certain proteins of the large 50S-ribosome subunit and one signal to a protein of the small 30S-ribosome subunit. In this case, these are proteins L29, L34, L36 and S22 with masses m/z=7274, 5381, 4365 or 5096. Since in particular the signals of the large subunit occur with high reliability in the mass spectra of Escherichia coli, they are especially suitable for identification. They were used as identified signals S_{id} for the synthetic reference spectrum REF₅ for Escherichia coli.

In an alternative or additional step S3 (FIG. 1), empirical signals Sₑₘ are determined from the measured reference spectra REF_{N} of the known microorganisms. The determination of the empirical signals Sₑₘ is done by comparing a number of mass spectra REF_{N}, which were recorded from the same strain, to one another, as well as by comparison with those of other organisms. In this case, such signals are determined to be characteristic of a microorganism that occur as frequently as possible in the mass spectra of the same organism and as rarely as possible in the mass spectra of another. For the frequency of an occurrence of a suitable empirical signal Sₑₘ, in this case a minimum value, for example >70% relative to all spectra of an organism, can be prescribed. Also, such a signal should have a minimum intensity that can be specified in advance to facilitate its differentiation from the background noise. The determination of empirical signals Sₑₘ in step S3 can be carried out in principle by visual spectra comparison by the user. It is preferably provided, however, to perform this step in an automated manner with the aid of suitable computer programs. Here, an algorithm that examines the measured natural reference spectra REF_{N} with respect to the above-mentioned criteria is suitable. Computer-supported processes, for example from infrared spectrometry, that are able to detect and to filter out frequently recurring signals with use of statistical algorithms are also known, however.

The identified and empirical signals S_{id} and Sₑₘ that are determined in steps S2 and S3 are combined in a subsequent step S4 to form synthetic reference spectra REF_{S}. In this case, a reference spectrum REFₛ is produced for each known microorganism.

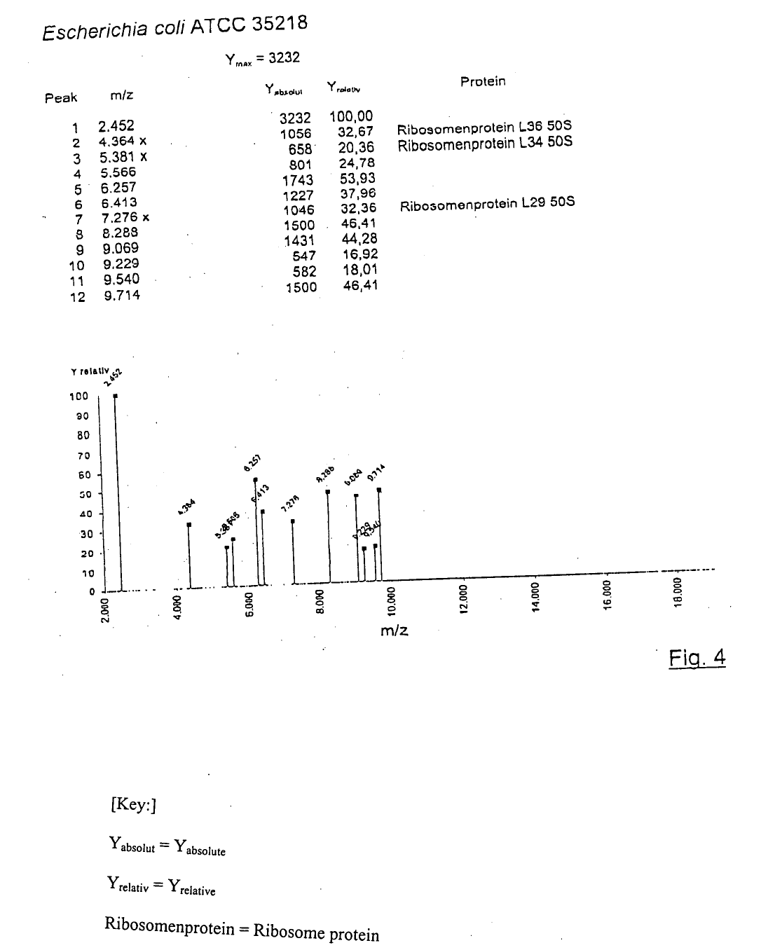

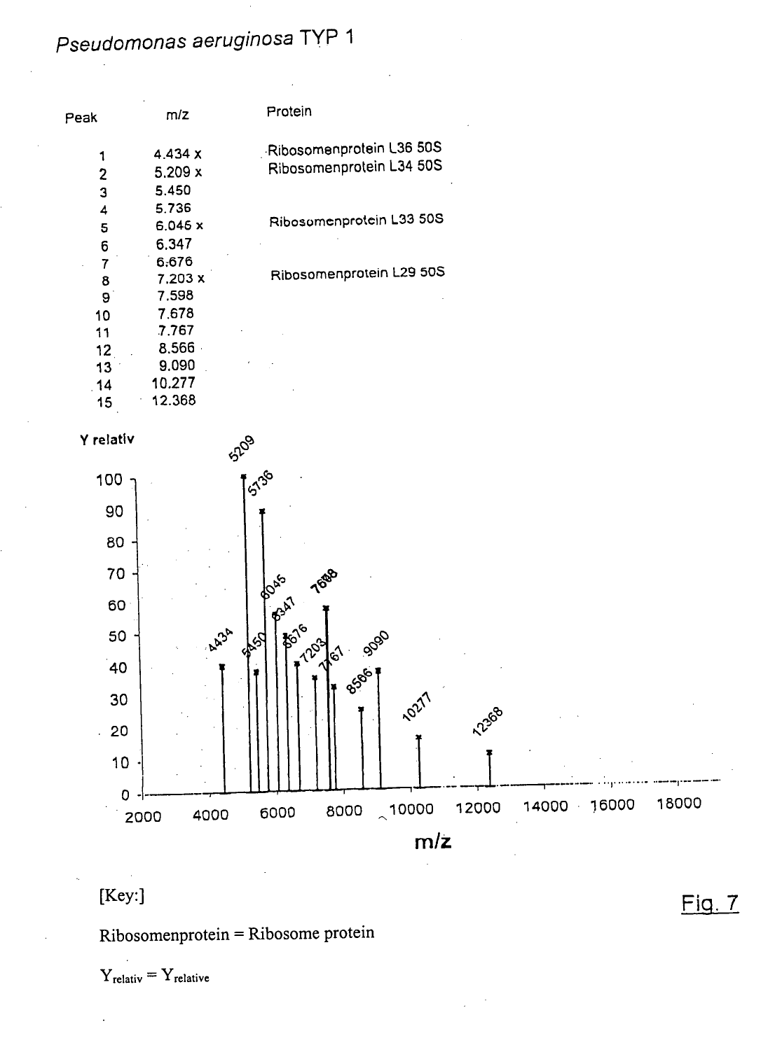

In FIGS. 3 to 9, such synthetic reference spectra REFₛ are shown by way of example for two Escherichia coli strains (ATCC 25922 and ATCC 35218), two Klebsiella species—namely Klebsiella oxycoca and Klebsiella pneumoniae, for Pseudomonas aeruginosa, Staphylococcus aureus as well as for the fungus Trichoderma reesei. In this case, the synthetic reference spectrum REF_{S} is shown in coordinate form in each case in the upper portion of each of FIGS. 3 to 9 (while partially eliminating the y-coordinates), while in the lower portion in each case, the graphic representation in the typical form of mass spectra is depicted. Synthetic reference spectra REF_{S} comprise ten to fifteen signals S in the examples shown and five signals in the case of the fungi T. reesei. In reference spectra REF_{S} of Escherichia coli (FIGS. 3 and 4) and of Pseudomonas aeruginosa (FIG. 7), the signals that are identified as proteins of the large 50 S-ribosome subunit are labeled S_{id}. In this case, signals that are allocated to proteins L29, L34 and L36 of the large subunit could be identified in each case for E. coli and for P. aeruginosa as well as in addition the signal that is allocated to protein L33 for P. aeruginosa. In the case of E. Coli ATCC 25922 (FIG. 3), it was also possible to relate a signal to protein S22 of the small 30 S-ribosome subunit.

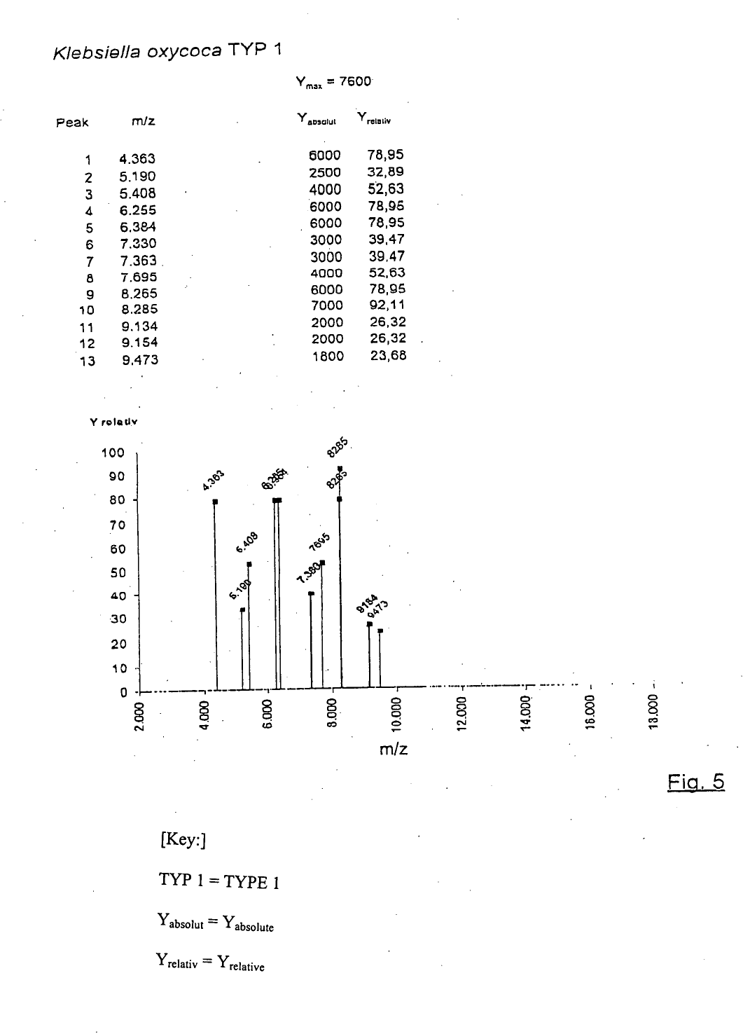

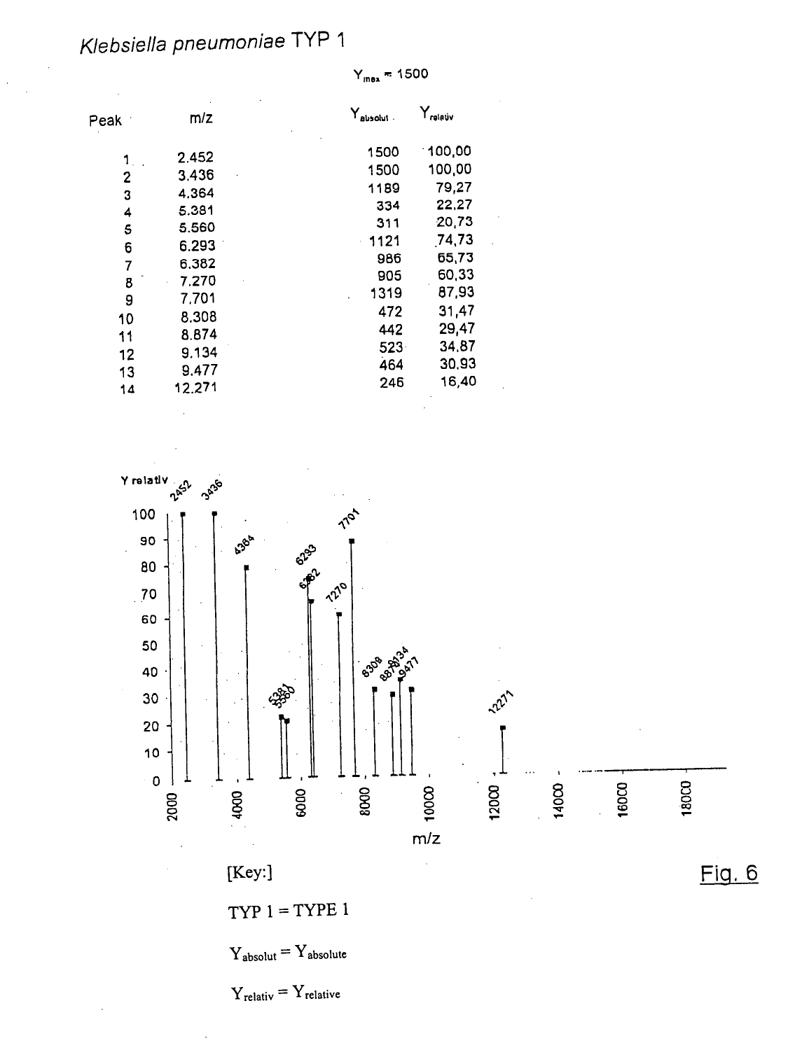

The differentiation of the two Escherichia coli strains from one another (FIGS. 3 and 4) is made possible mainly by those empirical signals Sₑₘ that are observed only in one of the two strains. A comparison of the synthetic reference spectra REF_{S} shown in FIGS. 3 and 4 shows that the process according to the invention is sensitive enough to make possible a differentiation of microorganisms even on the strain level. In addition, a differentiation between Escherichia coli on the one hand and the Klebsiella species (FIGS. 5 and 6) on the other hand is also made possible.

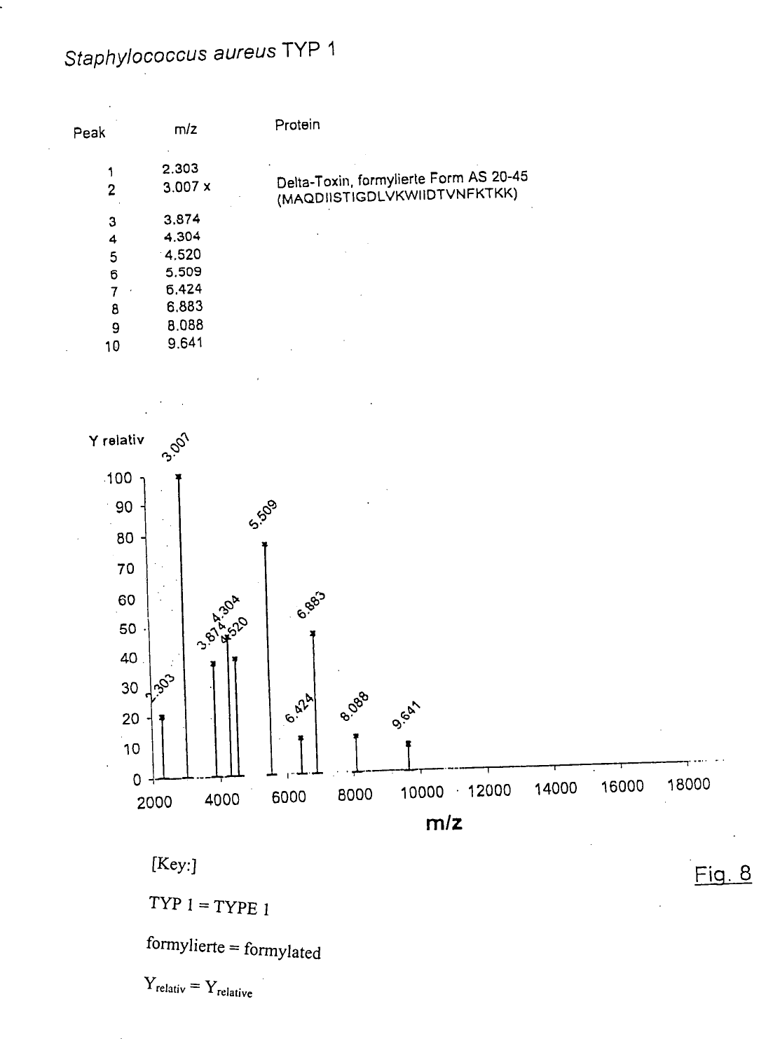

The example of Staphylococcus aureus, shown in FIG. 8, shows that others also can be identified as ribosome proteins and can be used for determining the organism. In this case, a signal could be allocated to a formylated peptide fragment of the delta toxin (delta hemolysine), which is extremely characteristic of this bacterium.

In the case of fungi, in addition to ribosomal proteins, in particular the hydrophobic structural proteins, hydrophobins have also proven advantageous for identifying the microorganism. Thus, for the fungus Trichoderma reesei (FIG. 9), three signals of the spectrum could be allocated to proteins hydrophobin I and II or to a fragment of hydrophobin II. These identified signals S_{id} can be used both for differentiation relative to bacteria that have no hydrophobins and for differentiation of various fungi from one another.

Further, difference spectra DIF in FIG. 1 are then produced in the production of synthetic reference spectra REF_{S} in step S5 by offsetting in each case two reference spectra REF_{S}. This step is illustrated in FIGS. 10a to 10d based on imaginary synthetic reference spectra of two closely related organisms, here genus 1 and genus 2 (FIGS. 10a and 10b). This step is preferably performed only for organisms that are very similar to one another, whereby the resulting difference spectra DIF facilitate the differentiation specifically of these organisms.

The offsetting of reference spectra REF_{S} is carried out by subtraction of one reference spectrum REF_{S} from the other in each case, whereby a signal that is present in both reference spectra REF_{S} is eliminated independently of the intensities. In this way, only signals that are not present in reference spectrum REF_{s,2} of genus 2 and vice versa remain in difference spectrum DIF₁₋₂ of genus 1. FIG. 10c shows the difference spectrum, “adjusted” by genus 2, for genus 1 DIF_{1,2}, and FIG. 10d shows the difference spectrum for genus 2 DIF₂₋₁ after “subtraction” of genus 1. Of course, in the offsetting of two signals, some device-specific and measurement-specific tolerances of the masses must be considered, such that as a result, signals S that are distinguished, for example, by one mass number are removed.

Signals that remain in the respective difference spectra DIF₁₋₂ and DIF₂₋₁ after the subtraction can be subjected to another selection. For example, it can be examined for spectrum DIF_{1,2} how frequently the signal occurs at the mass 5,500 in the natural original spectra of genus 1 (REF_{N,1}, not shown) and how frequently in the original spectra of genus 2 (REF_{N,2}, not shown). In this case, a signal is more likely eliminated the rarer it is in the natural eigen spectra and the more frequently it occurs in the natural foreign spectra, whereby intensities can also accordingly be considered.

Synthetic reference spectra REF_{S} that are produced in step S4 (FIG. 1) as well as difference spectra DIF that are produced in step S5 are combined in a subsequent step S6 in a data base DB. Within data base DB, the synthetic reference spectra REF_{S} can be arranged in a logical way, for example according to family, genus, type and strain. Data base DB can also comprise a statistic that reflects how a signal S has proven its value in the creation of synthetic reference spectra REF_{S} and/or difference spectra DIF with respect to frequency and specificity. This statistic can be used, for example, in the weighting of signal S in the identification of unknown organisms.

The design of data base DB according to the invention is shown in FIG. 11. The data base (optionally) comprises natural reference spectra REF_{N}, whereby for each organism i, j, and k, etc., a number of reference spectra REF_{S} are present. Data base DB also contains synthetic reference spectra REF_{S} that are determined for each known organism from the respective natural reference spectra REF_{N} that consist of a few discrete (identified and/or empirical) signals S. Finally, data base DB comprises difference spectra DIF, which were determined from, in each case, two synthetic reference spectra REFₛ of two microorganisms. In this case, it has proven adequate to produce only difference spectra DIF from those organisms that strongly resemble one another and are therefore difficult to differentiate by mass spectrometry according to experience.

The course of the process from S1 to S6 according to FIG. 1, i.e., the production of data base DB, must be performed in principle only once. It can then always be possible to use this data base DB then in the identification process according to the invention. Of course, data base DB can and should be broadened and updated constantly by reference spectra REFₛ and/or difference spectra DIF of additional microorganisms. It is conceivable, for example, to incorporate reference spectra REFₛ from mutants, especially those with altered resistance behavior and/or virulent behavior, if these strains yield characteristic signals. Also, based on the statistical evaluation of the analyzed samples, the existing reference spectra REFₛ should be continuously optimized, for example by receiving new signals S_{id} and Sₑₘ and/or by extinguishing signals.

The course of the actual identification process with use of the described data base DB is explained in FIG. 12. In step S7, the acquisition of a mass spectrum SAM of a microorganism that is to be identified is carried out. Advantageously, this sample spectrum SAM is measured under measuring conditions that are as identical as possible, especially the same matrix, like natural reference spectra REF_{N}.

Below (step S8), a similarity analysis of sample spectrum SAM with synthetic reference spectra REFₛ that are contained in data base DB is carried out in a first analysis step, whereby the sample spectrum is compared to each reference spectrum REF_{S}. This spectral adjustment can be carried out visually according to certain specified criteria, preferably with computer support, however. In this case, in principle, it is possible to use the same or similar algorithms, which were already used in step S3 to determine empirical signals Sₑₘ. By the simplicity of reference spectra REF_{S} that are used, the spectral adjustment can also be performed in step S8 with very simple algorithms, which are limited only to a comparison of signals S_{id}, Sₑₘ of synthetic reference spectra REF_{S} with sample spectrum SAM. Of course, tolerance criteria must be specified here that determine by how much a suitable signal S_{id}, Sₑₘ of sample spectrum SAM should deviate with respect to the mass and/or the intensity to be evaluated as similar. In addition, at this point, the weightings of individual signals S that are contained in the statistics can have an effect, whereby a signal S is all the more decisive for a positive identification the more often and more intensively it is present in the natural eigen spectra and the more rarely and weaker it is present in the natural foreign spectra. A minimum number of similar signals can also be specified for a positive identification.

In step S9, an output of a first result RES1 is carried out. This can consist either in that a clear correspondence of sample spectrum SAM with one of reference spectra REF_{S} was already recognized, with the result that the process is already completed at this point. If sample spectrum SAM, however, exhibits similarity with more than one synthetic reference spectrum REF_{S}, a number of suitable microorganisms are output as result REF1. In this case, a similarity analysis of sample spectrum SAM with difference spectra DIF that are contained in data base DB follows in step S10 as a second analysis step. If, in step S8, for example, a high similarity to reference spectra REF_{S} of organisms i and k is detected, an adjustment of sample spectrum SAM is carried out in step S10 with difference spectra DIF₁₋ₓ and DIFₖ₋₁ (cf. FIG. 11) that are produced between these organisms. The second analysis step according to the invention thus allows a more reliable allocation of an unknown organism to an organism from a group of organisms that are very similar to one another.

As final result RES2, the microorganism that is determined to be similar to sample spectrum SAM is output in step S11. Further, information on the degree of the similarity can also be output. As is frequently the case, in the sample of the unknown organism, this is a mixed culture and thus the process makes possible even the identification of several microorganisms that are present beside one another. In such a case, several hits are output as a result. If sample spectrum SAM does not correspond to any of the synthetic reference spectra REFₛ within the allowed tolerances, i.e., the unknown microorganism is not contained in data base DB, the latter is also a viable result.

In certain cases in which a microorganism cannot be identified or cannot be clearly identified, it can advantageously be provided that following step S11, an additional direct comparison to the measured “natural” reference spectra REF_{N} is performed. This procedure corresponds to the known procedure for identifying microorganisms by means of MALDI-TOF-MS and requires a data base DB, which in addition to the synthetic reference spectra REFₛ and the difference spectra DIF also comprises natural reference spectra REF_{N}.

By the simplicity and the concentrated information content of synthetic reference spectra REF_{S}, the process that is known according to the invention is distinguished by a significantly higher reliability that is still further increased by the second analysis step.

Legend

S1 Measurement of reference spectra

S2 Signal identification

S3 Determination of empirical signals

S4 Production of a synthetic reference spectrum

S5 Production of difference spectra

S6 Creation of a data base

S7 Detection of a sample spectrum

S8 Adjustment: Sample spectrum/synthetic reference spectrum

S9 Output of results 1

S10 Adjustment: Sample spectrum/difference spectra

S1 Output of results 2

DB Data base

DIF Difference spectrum

REF_{N} Natural reference spectrum

REFₛ Synthetic reference spectrum

RES1 Result from the first analysis step

RES2 Result from the second analysis step

SAM Sample spectrum

S_{id} Identified signal

Sₑₘ Empirical signal

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever. Also, any preceding examples can be repeated with similar success by substituting the generically or specifically described reactants and/or operating conditions of this invention for those used in such examples.

Throughout the specification and claims, all temperatures are set forth uncorrected in degrees Celsius and, all parts and percentages are by weight, unless otherwise indicated.

The entire disclosure of all applications, patents and publications, cited herein and of corresponding Germany application No. 103 00 743.1, filed Jan. 7, 2003, and U.S. Provisional Application Ser. No. 60/438,315, filed Jan. 7, 2003 are incorporated by reference herein.

From the foregoing description, one skilled in the art can easily ascertain the essential characteristics of this invention and, without departing from the spirit and scope thereof, can make various changes and modifications of the invention to adapt it to various usages and conditions.

1. Process for identifying microorganisms by means of mass spectrometry, whereby
(a) a data base (DB) is used, comprising
synthetic reference spectra (REF_{S}) of known microorganisms, which are formed by a combination of a number of signals (S) that are specific to the respective microorganism that is reduced relative to natural mass spectra (REF_{N}), as well as
difference spectra (DIF), which are formed by offsetting in each case two synthetic reference spectra (REF_{S}) of the known microorganisms,
(b) in a first analysis step, a similarity analysis of a sample mass spectrum (SAM) of a microorganism that is to be identified is performed with synthetic reference spectra (REF_{S}) that are contained in data base (DB), and
(c) in a second analysis step, a similarity analysis of sample spectrum (SAM) is performed with at least one portion of the difference spectra (DIF) that are contained in data base (DB).

2. Process according to claim 1, characterized in that difference spectra (DIF) are formed by subtraction of two synthetic reference spectra (REF_{S}) such that signals (S), which are present in two of reference spectra (REF_{S}) that are offset to one another, are eliminated independently of intensity.

3. Process according to claim 2, wherein signals (S), which remain after the subtraction in a difference spectrum (DIF) of a known microorganism, are selected and/or weighted by adjustment with natural mass spectra (REF_{N}) of this microorganism and/or with natural mass spectra (REF_{N}) of the microorganism that is offset with the latter.

4. Process according to claim 3, wherein the selection and/or weighting of signals (S) of difference spectrum (DIF) are selected based on their frequency and/or intensity in the natural mass spectra (REF_{N}) of the microorganism in question and/or based on their frequency and/or intensity in natural mass spectra (REF_{N}) of the microorganism that is offset with this microorganism.

5. Process according to claim 1, wherein as a result of the first analysis step, a number of synthetic reference spectra (REF_{S}) similar to sample spectrum (SAM) is determined, and the similarity analysis of the second step is performed only with difference spectra (DIF) that were obtained from the offsetting of synthetic reference spectra (REF_{S}) that are determined to be similar.

6. Process according to claim 5, wherein data base (DB) comprises only difference spectra (DIF) of microorganisms that have synthetic reference spectra (REF_{N}) that resemble one another.

7. Process according to claim 1, wherein signals (S) of a synthetic reference spectrum (REF_{S}) comprise at least one identified signal (S_{id}) that was attributed to a certain molecular cell component of the respective microorganism.

8. Process according to claim 7, wherein the cell component is a peptide, a protein, a ribonucleic acid and/or a lipid.

9. Process according to claim 7, wherein the microorganism is a bacterium and the cell component is a ribosomal protein.

10. Process according to claim 7, wherein the microorganism is a fungus, and the cell component is a structural protein, especially a hydrophobin, and/or a ribosomal protein.

11. Process according to claim 1, wherein signals (S) of a synthetic reference spectrum (REF_{S}) comprise at least one empirical signal (Sₑₘ), which was determined by comparison of mass spectra of known microorganisms to be specific for a microorganism.

12. Process according to claim 11, wherein the comparison is performed visually and/or with computer support.

13. Process according to claim 11, wherein as criteria for determining an empirical signal (Sₑₘ), a minimum frequency that can be specified in advance for an occurrence of the signal in a number of mass spectra of the same microorganism and a minimum intensity of the signal that can be specified in advance are specified.

14. Process according to claim 1, wherein a signal (S) is represented by a coordinate pair, consisting of a mass (m) or a mass-charge ratio (m/z) as x-coordinates and an absolute or relative intensity as y-coordinates.

15. Process according to claim 1, wherein the number of signals (S) of a synthetic reference spectrum (REF_{S}) is 1 to 50, especially 5 to 30.

16. Process according to claim 1, wherein the incorporation of mass spectra of non-pretreated cells is performed.

17. Process according to claim 1, wherein the similarity analysis of mass spectrum (SAM) of the microorganism that is to be identified with reference spectra (REF) that are contained in data base (DB) is limited to a comparison of signals (S) that are contained in reference spectra (REF).

18. Process according to claim 1, wherein the similarity analysis of mass spectrum (SAM) of the microorganism that is to be identified with difference spectra (DIF) that are contained in data base (DB) is limited to a comparison of signals (S) that are contained in difference spectra (DIF).

19. Process according to claim 1, wherein weightings are related to signals (S) that are contained in reference spectra (REF_{S}) for the similarity analysis.

20. Process according to claim 1, wherein all mass spectra are recorded with MALDI-TOF-MS.

21. Data base (DB) for implementing a process according to claim 1, comprising
(a) synthetic reference spectra (REF_{S}) of known microorganisms, containing a number of signals (S) specific to the respective microorganism that is reduced relative to natural mass spectra (REF_{N}), as well as
(b) difference spectra (DIF), resulting from an offsetting in each case of two synthetic reference spectra (REF_{S}) of the known microorganisms.

22. Data base (DB) according to claim 21 also comprising
(c) a number of natural mass spectra (REF_{N}) for each known microorganism.

23. Data base (DB) according to claim 22, wherein the number is at least 10.

24. Data base (DB) according to claim 21, wherein the natural mass spectra (REF_{N}) are MALDI-TOF mass spectra.

25. Use of a data base according to claim 21 for the identification of microorganisms by means of mass spectrometry, especially with MALDI-TOF-US.
